(19) United States
(12) Patent Application Publication
(10) Pub. No.: US 2015/0250554 A1
(43) Pub. Date: Sep. 10, 2015

(54) LUMINESCENT PATIENT CONNECTOR FOR PHYSIOLOGIC SIGNAL ACQUISITION

(71) Applicant: General Electric Company, Schenectady, NY (US)

(72) Inventors: Brian J. Young, Wauwatosa, WI (US); Corky Roe, Wauwatosa, WI (US)

(73) Assignee: GENERAL ELECTRIC COMPANY, Schenectady, NY (US)

(21) Appl. No.: 14/197,843

(22) Filed: Mar. 5, 2014

(51) Int. Cl. A61B 19/00; A61B 5/00; A61B 5/04; A61B 5/0408

(52) U.S. Cl. CPC A61B 19/5202; A61B 5/0408; A61B 5/742; A61B 5/7475; A61B 5/04012; A61B 2562/222; A61B 2562/08; A61B 2019/5204; A61B 2019/5206; A61B 2560/0214; A61B 2019/521; A61B 2562/226; A61B 2562/227

(57) ABSTRACT

A luminescent patient connector connectable to a device that acquires physiologic signals comprises a leadwire and a luminescent casing. The leadwire has a first end connectable to an electrode and a second end having a device connector configured to connect to an acquisition device for acquiring a patient physiological signal. The luminescent casing is around at least a portion of the leadwire and is configured to receive light input and permit the transmission of light across the length of the leadwire to illuminate at least the first end of the leadwire.

### BACKGROUND

Clinicians overseeing patient monitoring or performing diagnostic recordings of physiologic signals often have to work in low light conditions, such as in patient rooms at night when the lights are off. Turning on the lights to attach leadwires to a patient and/or check on a monitoring device can be disturbing to a patient. However, working in low light conditions is less than ideal for the clinician and can lead to mistakes, such as misplacement of electrodes.

Working in low light conditions can be especially problematic when a clinician needs to attach color-coded leadwires to a patient, for example according to certain guidelines set by national or international bodies. For instance, clinicians attaching electrocardiography (ECG) leadwires to a patient may need to follow color-coding guidelines set by the American Heart Association (AHA) system or the International Electrotechnical Commission (IEC). It can be difficult for the clinician to see the color coding on the ECG leadwires in low light conditions, thus further increasing the possibility of errors made by clinicians working in low light conditions.

### SUMMARY

The inventors of the present system and device recognize that it is preferable to find a way for clinicians to safely and effectively conduct patient monitoring in low light conditions. Accordingly, the present inventors invented the system disclosed herein having luminescent patient connectors that are visible and easily differentiated from each other in low light conditions.

In one embodiment, a luminescent patient connector connectable to a device that acquires physiologic signals comprises a leadwire and a luminescent casing. The leadwire has a first end connectable to an electrode and a second end having a device connector configured to connect to an acquisition device for acquiring a patient physiological signal. The luminescent casing is around at least a portion of the leadwire and is configured to receive light input and permit the transmission of light across the length of the leadwire to illuminate at least the first end of the leadwire.

In another embodiment, a luminescent patient connector connectable to a device that acquires physiologic signals comprises a leadwire and a light source. The leadwire has a first end connectable to an electrode and a second end having a device connector configured to connect to an acquisition device for acquiring a patient physiological signal. The light source is located between the first end and the second end of the leadwire.

In yet another embodiment, a system for acquiring physiologic signals from a patient comprises an acquisition device configured to acquire a patient physiological signal and a lead connection port in the acquisition device. The system further comprises a light source that emits light into a luminescent patient connector, wherein the luminescent patient connector comprises a leadwire having a first end connectable to an electrode and a second end having a device connector configured to connect to the acquisition device. In one embodiment of the system, the light source may be located in the luminescent patient connector. Alternatively or additionally, the light source may be located in the acquisition device and configured to emit light into the luminescent patient connector.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 depicts one embodiment of a patient monitoring system including an acquisition device and a luminescent patient connector having a light source therein.

FIG. 2 depicts another embodiment of a patient monitoring system including an acquisition device and a luminescent patient connector having a light source therein.

FIG. 3 depicts an embodiment of a patient monitoring system including an acquisition device having a light source therein and a luminescent patient connector.

FIG. 4 depicts another embodiment of a patient monitoring system including an acquisition device having a light source therein and a luminescent patient connector.

### DETAILED DESCRIPTION

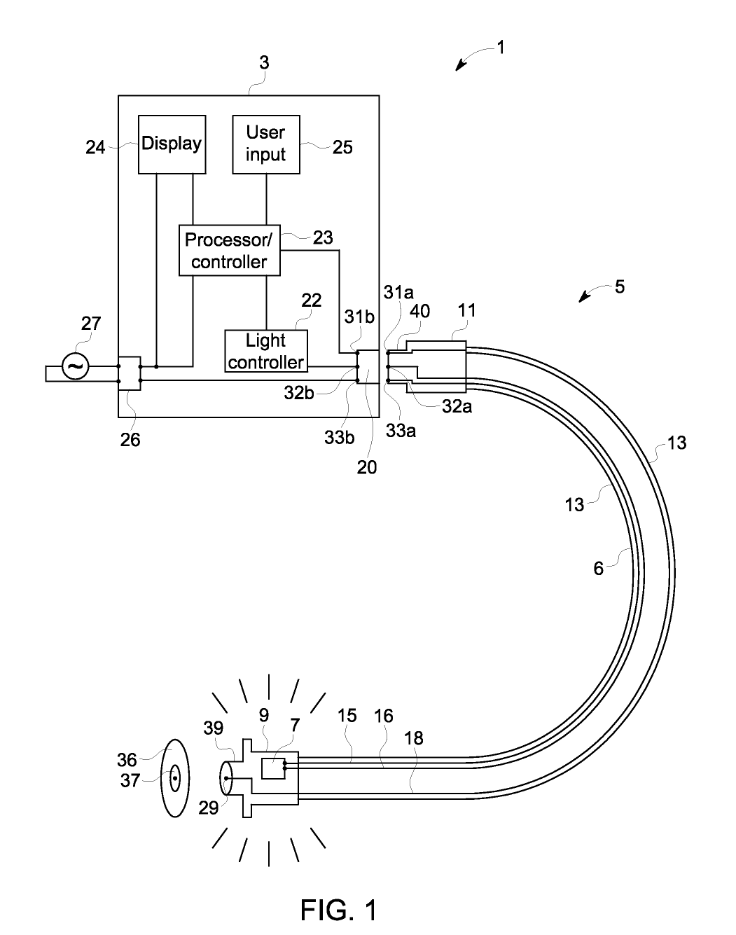

FIG. 1 demonstrates an embodiment of such a system having an acquisition device 3 and a luminescent patient connector 5. The acquisition device 3 acquires one or more patient physiological signals. For example, the acquisition device 3 may be a cardiac patient monitor, a diagnostic electrocardiograph, a multi-parameter patient monitor, or a neurological monitoring device, such as an electroencephalograph (EEG), or any other device that acquires and/or monitors a physiological signal from a patient. In the embodiment of FIG. 1, the acquisition device 3 comprises a processor/controller 23 that controls a display 24, a user input 25 and a light controller 22. The processor/controller 23 receives a physiological signal from a patient through the luminescent patient connector 5. The processor/controller 23 may be powered from power source 27, such as through the power source regulator/controller 26. The power source regulator/controller 26 may operate to control and/or limit power delivery to one or more of the various components of the monitoring system 1. The power source 27 may be any power source known in the art, including power from a power grid, AC power, DC power, battery power, generator power, etc.

The acquisition device 3 contains at least one lead connection port 20 that receives, or connects to, a luminescent patient connector 5. It is contemplated that embodiments of the system 1 may include acquisition devices 3 with any number of lead connection ports 20 and may include any number of luminescent patient connectors 5. In the embodiment of FIG. 1, the luminescent patient connector 5 is comprised of a leadwire 6 having a first end 39 and a second end 40. The first end 39 of the leadwire 6 has an electrode connector 9 that connects to, is connected to, or is connectable to, an electrode attachable to a patient to record a physiological signal. The opposite end, the second end 40 of the leadwire 6, has a device connector that connects to the acquisition device 3. The depicted luminescent patient connector 5 contains a light source 7 in the electrode connector 9. The light source may be powered through the acquisition device 3 and controlled by the light controller 22 in the acquisition device 3. The light source 7 in the embodiment of FIG. 1 operates to illuminate the electrode end of the luminescent patient connector 5 to provide visibility and identification of the lead and electrode in low light conditions. Thereby, a clinician may be enabled to position and/or check electrode placement and/or lead connection on a patient in low light conditions, such as in a hospital room at night.

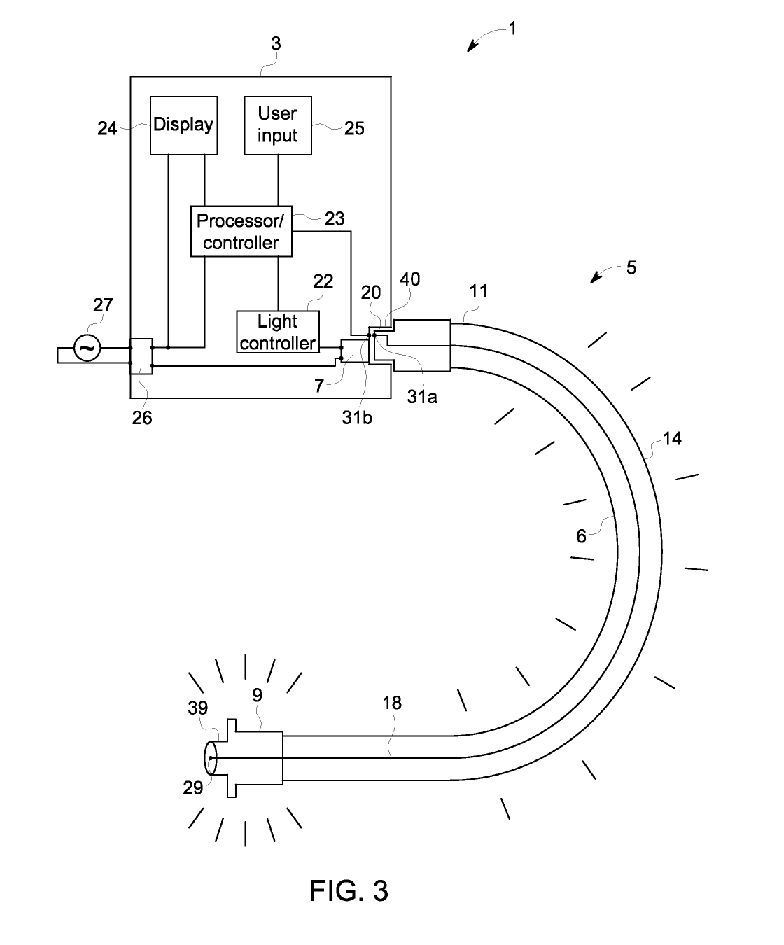

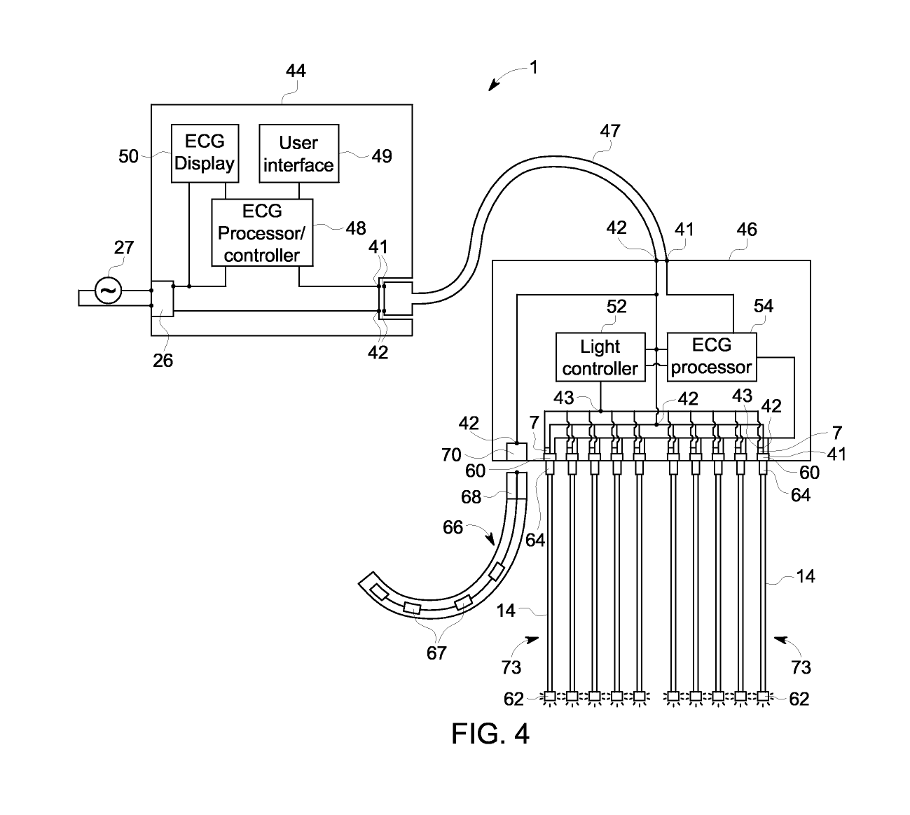

The light source 7 may be placed or embedded anywhere within the physiological signal acquisition system 1 wherein light from the light source 7 can be transmitted into the luminescent patient connector 5 to illuminate at least a portion of the lead. The light source may be located anywhere within the luminescent patient connector 5, itself, or may be located in the acquisition device 3 (see, e.g., FIGS. 3-4). The light source may illuminate any portion of the luminescent patient connector 5, including the device connector 11, the electrode connector 9, the casings 13 around the leadwire, and/or all of the above such that the entire leadwire 6 is illuminated. The light source 7 in the embodiment of FIG. 1 may be any light source capable of being housed in the electrode connector 9 of the luminescent patient connector 5. For example, the light source 7 may be a light emitting diode (LED). In other embodiments, the light source 7 may be a small and thin light emitting display constructed from a technology such as an organic LED (OLED) or may be a light source such as a LED in the connector combined with a non-light emitting display such as super low power electronic paper constructed from a technology such as an electrophoresis display.

The light controller 22 acts to control the light source 7, for example by turning on and off the light source as appropriate. In one embodiment, the light source 7 may be programmable, such as to vary in color, to blink, or to turn on and off in response to certain conditions or instructions from the light controller 22. For example, the light source 7 may be a multicolor LED that can selectably emit light of more than one color, or multiple LEDs of different colors, and the light controller 22 may control the color of the light source 7. In the embodiment of FIG. 1, the light controller 22 is connected to and controlled by the processor/controller 23. For example, the processor/controller 23 may control the light controller 22 in response to user input through the user input 25. In other embodiments, the functions of the light controller 22 may be integrated into the processor/controller 23, such that the processor/controller 23 controls the light source 7. In still other embodiments, the light controller 22 may operate independently of the processor/controller 23. Likewise, the light controller 22 may be powered through the processor/controller 23, or it may be separately connected to the power source regulator/controller 26.

The light controller 22 controls the light source 7 via the control connection 16. In other words, a signal from the light controller 22 runs through the light controller connection point 32B in the lead connection port 20 to the connection point 32A in the device connector 11, which connects to the control connection 16 that leads to the light source 7. The light source 7 may be powered similarly by the power source controller 26 in the acquisition device 3. The power connects through the power connection point 33B in the lead connection port 20 to the power connection point 33A in the device connector 11, which connects to the power connection 15 attached to the light source 7. In other embodiments, the light source 7 may be powered by other means, such as by a small battery embedded somewhere within the luminescent patient connector 5, such as in the device connector 11, the electrode connector 9, or the electrode 36.

In the embodiment of FIG. 1, the leadwire 6 has an electrode connector 9 configured to connect to the snap 37 on the electrode 36. In that embodiment, the electrical connection point 29 on the electrode connector 9 electrically connects to the snap 37 on the electrode 36. In other embodiments, the electrode connector 9 may connect to the electrode 36 by any means known in the art. Alternatively, the electrode connector 9 may be permanently connected to an electrode such that the luminescent patient connector 5 includes an electrode attachable to a patient.

Returning to the embodiment of FIG. 1, electrical signals may be recorded from a patient when the electrode 36 is connected to that patient. The physiological signals are then transferred via the signal transmission line 18 to the connection point 31A of the device connector 11. When the device connector 11 is connected to the acquisition device 3, connection point 3 IA of the device connector electrically connects to connection point 31B in the lead connection port 20, and thereby physiological signals transmitted through transmission line 18 are transmitted to the processor 23. In another embodiment, the electrode connector 7 maybe permanently connected to an electrode. In such an embodiment, the luminescent patient connector 5 may be a single-use lead and may be disposable. In other embodiments, the luminescent patient connector 5 may be connectable to an electrode by any means known in the art, which may be permanent or removable connection means. For example, the electrode connector 9 may comprise any means or device for connecting to any electrode, such as an alligator clip, a snap, a threaded device, a conductive adhesive, etc.

The luminescent patient connector 5 for FIG. 1 is covered in a casing 13, which may be any casing described herein, such as the luminescent casing described below, or may be any casing known in the art suitable for covering a leadwire or patient connector. In one embodiment, the casing 13 may be comprised, at least in part, of a luminescent casing covering at least a portion of the leadwire, wherein the luminescent casing is configured to receive light from the light source 7 and transmit that light to illuminate at least a portion of the luminescent patient connector. In one exemplary embodiment, the electrode connector 9 containing the light source 7 may be comprised at least partially of material designed to diffuse and/or emit the light from the light source 7, and thus to be illuminated, or “glow”, when the light source 7 is on. In another exemplary embodiment, the luminescent patient connector 5 may have a luminescent casing around the leadwire 6 that transmits the light emitted by the light source 7 from the first end 39 to the second end 40 of the luminescent patient connector 5. In still other embodiments, the casing 13 may not have any luminescent properties and may be any casing known in the art.

The luminescent casing embodiment of the casing 13 may be, for example, casing with fiber optic properties that transmit and/or diffuse and emit the light from the light source across the luminescent patient connector 5. For example, the luminescent casing may be comprised of one or more end-emitting fiber optic fibers that transmit the light from the light source 7 in the electrode connector 9 to the device connector 11, which may be designed to diffuse and emit the light transported by the luminescent casing and thus to glow. Likewise, the electrode connector 9 housing the light source 7 may also be designed to emit at least some of the light from the light source 7 so as to also glow when the light source is on. In still other embodiments, the casing 13 may be a luminescent casing comprised of edge-emitting fiber optic fibers that transmit and emit the light from the light source 7. In such an embodiment, the casing 13 may emit light across the entire length of the leadwire 6, and thus some or all of the length of the leadwire may glow.

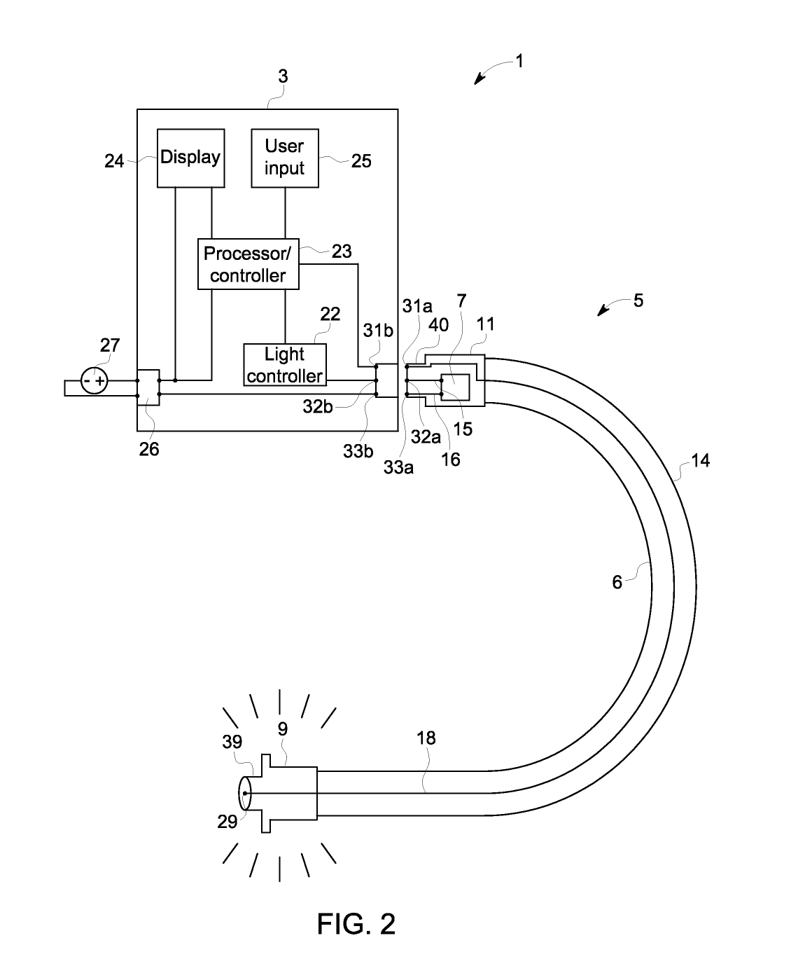

Alternatively or in addition to the embodiment of FIG. 1, a light source 7 may also be located in the device connector 11 of the luminescent patient connector 5. As depicted in FIG. 2, the leadwire may be covered in a luminescent casing 14 that transmits the light from the light source 7 to the electrode connector 9 so that the electrode connector 9 is illuminated. The luminescent casing 14 may have fiber optic properties that permit the transmission of the light from the light source 7 to the electrode connector 9. The electrode connector 9 may have light diffusing and emitting properties so that at least a portion of the electrode connector 9 lights up as a result of the light transmission through the luminescent casing 14. In one embodiment, the luminescent casing may be comprised of one or more end-emitting fiber optic fibers.

In the embodiment of FIG. 2, the light source 7 may be any light source. By way of example, the light source 7 may be a fiber optic illuminator configured to efficiently focus light into the luminescent casing 14 and to be the source of light emitted by the electrode connector 9. In another embodiment, the light source 7 may be configured to emit light into the device connector 11 in addition to the luminescent casing 14, such that both the electrode connector 9 and the device connector 11 illuminate when the light source is on. In still other embodiments additional portions of the luminescent patient connector 5 may illuminate when the light source 7 is on. The light source 7 may be any light source or any fiber optic illuminator known in the art. For example, the light source 7 may consist of a light bulb, such as a small quartz halogen lamp, xenon metal halide lamp, or an incandescent or other bulb. Alternatively, the light source 7 may be an LED, or multiple LEDs, which may be used to efficiently couple light into the luminescent casing.

The luminescent casing may cover all or a portion of the leadwire. For example, the luminescent casing 14 may comprise a strip along the length of the leadwire sufficient to conduct and/or transmit light along a portion thereof. In one embodiment, the luminescent casing 14 may be made of a plastic material that houses one or more end-emitting fibers, or end-glow fibers. The plastic material may be light-absorbing material, such as an opaque plastic material, or it may be translucent. In such an embodiment, only the electrode connector 9 may illuminate when the light source 7 is on. In another embodiment, the luminescent casing 14 may comprise one or more edge-emitting fiber optic fibers that emit light along at least portions of the length of the fiber, which may cause an additional portion of the leadwire 6 to illuminate. In one exemplary embodiment, the luminescent casing 14 may be comprised of a translucent material, such as a plastic, that diffuses light from the one or more edge-emitting fibers embedded therein, thereby causing the entire luminescent casing 14 surrounding the luminescent patient connector 5 to illuminate. In another embodiment, the luminescent casing 14 may comprise an opaque material that does not permit transmission of light. In such an embodiment containing edge-emitting fibers, the luminescent casing 14 may be configured to allow the edge-emitting fibers to diffuse and emit light along the length of the leadwire such that the one or more edge-emitting fibers product an illuminated line along the length of the luminescent patient connector 5 when the light source 7 is on. In still other embodiments, the luminescent casing 14 may be comprised of a sequence of LEDs or other small electronically controlled light-emitting sources along the length of the leadwire 6, thereby creating a luminescent casing for the leadwire by using LEDs rather than fiber optics.

In still other embodiments, the light source 7 may be located in the acquisition device 3 and positioned therein so that it transmits light into at least a portion of the luminescent patient connector 5. In the embodiment of FIG. 3, the light source 7 is located in the lead connection port 20. There, light source 7 is configured to transmit light into the luminescent patient connector 5. As described above, depending on the configuration of the luminescent patient connector, any portion of the luminescent patient connector 5 may be configured to illuminate and thus emit the light from the light source 7. In the embodiment of FIG. 3, the leadwire 6 is covered in a luminescent casing 14 configured to emit light along at least a portion of the length of the leadwire 6 including the electrode connector 9. Thus, as described above, the luminescent casing 14 of FIG. 3 may be any casing that permits transmission of the light from the light source 7 to the electrode connector 9, while also permitting the emission of at least some of the light from the light source 7 along at least an additional portion of the length of the leadwire 6. In one such embodiment, the entire luminescent patient connector 5 may illuminate from the first end 39 to the second end 40 of the leadwire 6. In another such embodiment, the entire leadwire 6 except for the device connector 11 may illuminate. In still other embodiments within the scope of FIG. 3, the end connector 9 may illuminate and portions of the length of the leadwire 6 may illuminate.

The light source 7 of FIG. 3, which is positioned in the lead connection port 20, may be any light source capable of transmitting light into the luminescent patient connector 5. For example, the light source may be a fiber optic illuminator consisting of a light source that efficiently focuses light into the luminescent casing 14. For example, the light source 7 may be an incandescent bulb, a quartz halogen lamp or a xenon metal halide lamp may be used to create a bright light source. In another embodiment, a smaller light source such as an LED may be used. In still another embodiment, multiple LEDs may be employed. The light source 7 may be configured to compliment or coordinate with the configuration of the luminescent casing 14 so that the light from the light source 7 is directed into the luminescent casing 14 to effectively and efficiently create the desired illumination of the luminescent patient connector 5.

Turning to FIG. 4, the acquisition device may be comprised of any device for acquiring a physiologic signal from a patient, such as an electrocardiograph (ECG), connected to an acquisition device, such as an ECG acquisition device 46. In the embodiment of FIG. 4, the ECG monitor 44 is comprised of an ECG processor/controller 48 connected to an ECG display 50 and a user interface 49. The ECG also connects to a power source 27 via power source regulator/controller 26. The ECG monitor 44 connects to an ECG acquisition module 46 via connection cord 47. The ECG acquisition module 46 contains an ECG processor 54 that receives and processes analog or digital cardiac signals from a patient via one or more luminescent cardiac leads 73.

Additionally, the patient monitoring system 1 of FIG. 4 also contains an additional luminescent element 66 attachable to the ECG acquisition module 46. Specifically, the additional illumination element 66 may have a device connector 68 that connects to the connector port 70 in the ECG acquisition module 46. The additional luminescent element 66 has one or more additional light sources 67 and is configured to provide a source of additional illumination, for example to allow a clinician to illuminate an area of a patient where the clinician might be working, for example to attach ECG leads to a patient and/or check the status of ECG leads already attached to a patient. The additional illumination element 66 may be provided separate from the luminescent patient connectors, such as the luminescent cardiac leads 73, or it may be provided attached to or in conjunction with one or more monitoring leads.

In the embodiment of FIG. 4, the ECG acquisition module 46 contains ten lead connection ports 60 that connect to ten luminescent cardiac leads 73. Similar to the luminescent patient connector 5 described above, each luminescent cardiac lead 73 may comprise a device connector 64 that connects the luminescent cardiac lead 73 to the ECG acquisition module 46 and an electrode connector 62 that connects the luminescent cardiac lead 73 to an electrode. The luminescent cardiac lead 73 conducts the cardiac signal from an electrode attached to a patient to the data transmission connection point 31 in the lead connection port 60 of the ECG acquisition module 46. From there, the patient cardiac signal is processed by the ECG processor 54. For example, the ECG processor 54 may convert the analog patient cardiac signal to a digital signal and may then transmit a digital signal to the ECG processor/controller 48 of the ECG monitor 44. Alternatively or additionally, the ECG processor 54 in the acquisition device 46 may perform other functions, such as processing the cardiac data to determine whether all electrodes are connected to and properly placed on the patient. Likewise, the ECG processor may conduct additional processing of the cardiac data, for example to detect an alarm condition or to perform diagnostic analysis. In still other embodiments, the ECG processor 54 in the acquisition device 46 may provide only minimal processing, for example to eliminate noise and/or amplify the cardiac signal before transmission to the ECG monitor 44.

In still other embodiments, the ECG acquisition module 46 may not contain any processor 64 and/or any control device and may simply conduct the analog cardiac signal from the patient to the ECG processor/controller 48 in the ECG monitor 44. In such an embodiment the acquisition module would be a simple device configured to provide a connection point for the luminescent patient connectors, such as those in the depicted embodiments, and to passively transmit, or conduct, the signals from the leads to the processor housed in the patient monitor.

In the embodiment of FIG. 4, the acquisition device 46 has a light source 7 in each lead connection port 60. Each light source 7 may be controlled by the light controller 52 in the ECG acquisition module 46. For example, the light controller 52 may selectively turn on and off one or more of the light sources 7, and/or the light controller 52 may control the color and/or intensity of each light source 7. For example, the light controller 52 may control the light source 7 based on instructions from the ECG processor 54. In one such embodiment, the light controller 52 may control the light source 7 to illuminate the luminescent cardiac lead 73 based on whether the lead is property connected to the patient. In one such embodiment, the light controller 52 may blink the light source 57, which would cause blinking in the luminescent cardiac lead 73, to indicate when a particular lead is not connected or is improperly connected. In still other embodiments, the light controller 52 may be programmed to selectively turn on or off the light source 7 in response to a proper or improper connection to the patient. In still other embodiments, the light controller may be responsive to an instruction received from the clinician through the user interface 49 in the ECG monitor 44. For example, the clinician may use the interface 49 to turn on or off the light source 7, or to change the color of illumination intensity of the light source 7. In yet another embodiment not depicted, the ECG acquisition module 46 may have a separate user interface whereby a clinician or a user can control one or more of the light sources and/or the additional luminescent element.

The light sources 7 in an embodiment like that of FIG. 4 may be of different colors so as to provide luminescent electrodes of varying colors. For example, the light sources 7 may be sufficiently variable such that each light source 7 in each of the lead connection ports 60 can illuminate in a different color from the other light sources in the other ports. Such an embodiment allows for compliance with the regulations of the American Heart Association (AHA) and the International Electrotechnical Commission (IEC) regarding color coding or labeling of cardiac leads. In other words, the electrodes may be illuminable in colors or illuminate character labeling that comply with relevant guidelines. In a related embodiment, the light source 7 may be comprised of a multicolor LED, or of multiple LEDs of different colors. In such an embodiment, the light controller 52 may control the one or more light sources 7 to selectably or variably illuminate the luminescent cardiac lead 73 in different colors depending on factors such as lead type, lead placement, light conditions, and user preference. For example, in the context of the embodiment in FIG. 4, each of the ten light sources 7 may provide a different light color than the other nine light sources. Thereby, each of the ten luminescent cardiac leads 73 may be illuminated in a different color.

The light controller 52 may be configured to be responsive to the ECG processor 54 and/or to the ECG processor/controller 48 and the ECG monitor 44. In the embodiment of FIG. 4, the light controller 52 communicates with the ECG processor 54 within the ECG acquisition module 46.

The one or more light sources 7 in the acquisition module 46 of FIG. 4 are powered through the ECG monitor 44. Specifically, the light sources 7 are connected through power connection point 42 through the ECG acquisition device 46, into the ECG monitor 44 and back to the power source regulator/controller 26. Likewise, the additional luminescent element 66 is also powered through one or more of the control devices or through the power source regulator/controller 26 in the ECG monitor 44.

The additional luminescent element 66 may be any device capable of providing additional illumination to an area of a patient. For example, in the embodiment of FIG. 4, the additional luminescent element is a light cord having multiple additional light sources 67 connected in parallel or in series to the power connection point 42 in the connection port 70 for the additional luminescent element 66. In such an embodiment, the light cord may be placed on or near the work area on the patient to provide additional illumination to enable a clinician to work in that area on the patient in low light conditions. For example, the light cord could be placed on or near the patient's chest area to enable the clinician to see the chest are while connecting or checking ECG leads in low light conditions.

In other embodiments, the additional luminescent element may be an extension device with one or more lights at the end thereof that could be focused on the work area of the patient. Preferably, the additional luminescent element 66 would be flexible such that it can be maneuvered to illuminate a particular area, such as on a patient, and could be selectively connected and disconnected from the patient monitoring system 1 depending on need.

The additional light source 67 may be any light source known in the art. In one embodiment, the additional light source 67 is one or more LEDs, such as white LEDs, that provide good illumination while requiring only minimal power. In other embodiments, the additional light source 67 may be an incandescent bulb, or other type of light bulb.

It should be understood that the additional illumination element 66 may be included in any embodiment of the patient monitoring system 1 disclosed or described herein. For example, the patient system 1 may be configured such that the additional illumination element 66 is used in conjunction with standard leadwires or patient connections available in the patient monitoring industry. Furthermore, the additional illumination element 66 may be incorporated into any type of patient monitoring device. In such embodiments, the additional illumination element 66 may be used to illuminate the workspace sufficiently so that the clinician can adequately see the standard leadwires, which are non-illuminating. For example, in a preferred embodiment, the additional illumination element 66 may be capable of providing sufficient light so that a clinician in a dimly-lit or dark room can see the color-coding and/or other identification insignia on each leadwire in their workspace.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A luminescent patient connector connectable to a device that acquires physiologic signals, the luminescent patient connector comprising:
a leadwire having a first end connectable to an electrode and a second end having a device connector configured to connect to an acquisition device for acquiring a patient physiological signal; and
a luminescent casing around at least a portion of the leadwire configured to receive light input and permit the transmission of light across the length of the leadwire to illuminate at least the first end of the leadwire.

2. The luminescent patient connector of claim 1 wherein the first end of the leadwire has an electrode connector configured to attach to an electrode and to illuminate as a result of light input at the second end of the leadwire.

3. The luminescent patient connector of claim 1 wherein the luminescent casing is configured to illuminate the length of the leadwire as a result of light input at the second end of the leadwire.

4. The luminescent patient connector of claim 1 wherein the luminescent casing is comprised of one or more optical fibers, and wherein the optical fibers are comprised of end-emitting fibers and/or edge-emitting fibers.

5. A luminescent patient connector connectable to a device that acquires physiologic signals, the luminescent patient connector comprising:
a leadwire having a first end connectable to an electrode and a second end having a device connector configured to connect to an acquisition device for acquiring a patient physiological signal;
a light source located between the first end and the second end of the leadwire.

6. The luminescent patient connector of claim 5 wherein the light source is in the device connector and is configured such that the light source is powered by the acquisition device; and
wherein the luminescent patient connector further comprises a luminescent casing around at least a portion of the leadwire, wherein the luminescent casing is configured to receive light from the light source and to illuminate at least a portion of the luminescent patient connector.

7. The luminescent patient connector of claim 5 wherein the first end of the leadwire has an electrode connector configured to attach to an electrode, and wherein the light source is in the electrode connector and is configured such that such that the light source is powered by the acquisition device.

8. The luminescent patient connector of claim 5 further comprising a luminescent casing around at least a portion of the leadwire configured to receive light from the light source and to illuminate at least a portion of the leadwire.

9. The luminescent patient connector of claim 5 wherein the light source is a light emitting diode (LED).

10. A system for acquiring physiologic signals for a patient comprising:
an acquisition device configured to acquire a patient physiological signal;
a lead connection port in the acquisition device;
a light source that emits light into a luminescent patient connector; and
wherein the luminescent patient connector comprises a leadwire having a first end connectable to an electrode and a second end having a device connector configured to connect to the acquisition device.

11. The patient monitoring system of claim 10 wherein the first end of the leadwire has an electrode connector configured to attach to an electrode, and wherein the light source is positioned in the electrode connector.

12. The patient monitoring system of claim 10 wherein the luminescent patient connector further comprises a luminescent casing around at least a portion of the leadwire, wherein the luminescent casing is configured to receive light from the light source and to illuminate at least a portion of the luminescent patient connector.

13. The patient monitoring system of claim 12 wherein the light source is positioned in the connection port of the acquisition device.

14. The patient monitoring system of claim 13 further comprising:
two or more connection ports in the acquisition device, each configured to receive a device connector of a luminescent patient connector and each having a light source;
wherein each light source emits a different colored light from the other light sources in each of the two or more connection ports.

15. The patient monitoring system of claim 14, wherein each of the light sources is capable of emitting multiple light colors, and wherein each light source is connected to a controller in the acquisition device that controls the color of each light source.

16. The patient monitoring system of claim 10 wherein the acquisition device is a cardiograph, and wherein:
the lead connection port in the cardiograph is configured to receive a device connector of a luminescent electrocardiograph lead; and
the light source in the connection port emits light into the luminescent electrocardiograph lead.

17. The patient monitoring system of claim 10 wherein the acquisition device is an acquisition module connected to a cardiograph, and wherein:
the lead connection port in the acquisition module is configured to receive a device connector of a luminescent electrocardiograph lead; and
the light source in the connection port emits light into the luminescent electrocardiograph lead.

18. The patient monitoring system of claim 10 wherein the light source is a light emitting diode (LED), a quartz halogen lamp, or xenon metal halide lamp.

19. The patient monitoring system of claim 10 further comprising an additional luminescent element connectable to the acquisition device, wherein the additional luminescent element contains one or more additional light sources and serves to illuminate an area.

20. The patient monitoring system of claim 19 wherein the additional luminescent element is a light cord containing multiple LEDs along its length, wherein the light cord is configured such that the LEDs are powered by the acquisition device.
